(19) United States
(12) Patent Application Publication
(10) Pub. No.: US 2007/0172510 A1
(43) Pub. Date: Jul. 26, 2007

(54) Energy drink

(76) Inventor: Marc R. Melton, Encinitas, CA (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 11/338,604

(22) Filed: Jan. 23, 2006

(51) Int. Cl. A61K 36/54; A61K 31/522; A61K 36/254; A61K 36/899; A61K 36/48

(52) U.S. Cl. 424/439; 424/725; 424/743; 424/739; 424/757; 424/750; 424/773; 424/776; 514/263.31; 424/728; 514/567

(57) ABSTRACT

Disclosed are nutritional energy drinks and supplements. Also disclosed are methods of making nutritional drinks. The drinks provide energy to the body by refueling the body's fuel tank. The drinks comprise a core formula, to which additional ingredients are added to make a energy, performance or diet enhancing formula. The drinks may be prepared as a tea or a ready made drink.

### TECHNICAL FIELD

The present invention relates generally to the field of nutritional drinks and supplements. More particularly, this invention pertains to an improved energy drink that provides energy to the body by refueling the body's fuel tank, and to methods for making such energy drinks.

### BACKGROUND

The demand for beverages that help to enhance the mental and physical wellness of drinkers has increased dramatically. Many different types of sport drinks and energy drinks have been marketed in recent years that claimed to provide energy and nutrients to the drinker. For example, many of these drinks rely on stimulants such as added caffeine, sugar or ephedra to boost a drinker's energy level. Also, many of these energy drinks merely assist in the release of energy already stored in the drinkers system without providing a new source of energy. An analogy that can be made of this situation is to a car's fuel system. Current energy drinks help to squeeze usage out of the last remaining “fuel” in the body's “fuel” tank. But they do not add more “fuel”. Thus, these energy drinks supply a short term energy boost but leave the body depleted and in a worse condition. In traditional Chinese medicine, chi is the vital force that flows throughout the body. Energy drinks that do not provide more fuel to the body give a “false chi” —the illusion of energy.

Another difficulty is that many drinks provide the active ingredients in such small quantities as to make them little more than fluff for marketing purposes. Therefore, there is a need for a better nutritional drink.

### SUMMARY OF THE INVENTION

The present invention provides nutritional drinks that provide energy to the body by refueling the body's fuel tank. The energy drinks disclosed comprise one or more nutraceutials and a source of natural caffeine. The caffeine assists in the delivery of the nutraceutials to the blood stream. Preferably, at least one of the nutraceutials is a nootropic.

The nutritional drinks comprise a core formula to which other enhancing ingredients can be added to provide drinks with energy enhancing, performance enhancing, or diet aid properties. The core formula and other formulas may also be modified to provide a coffee additive supplement for adding to prepared coffee. The drink formula can be prepared as a tea for brewing, or as a ready to drink beverage. When prepared as a tea, the drink ingredients are placed in a tea bag according to the density of the ingredients. The tea is preferably brewed in a two step process, to enhance the extraction of active ingredients from the drink mix.

### BRIEF DESCRIPTION OF THE DRAWINGS

Non-limiting and non-exhaustive preferred embodiments of the present invention are described with references to the following figures wherein like reference numerals refer to like parts throughout the various views unless otherwise specified.

FIG. 1 is a chart illustrating the composition of various formulations of the present invention.

FIG. 2 is a flowchart outlining process of making embodiments of the energy drink of the present invention.

### DETAILED DESCRIPTION OF THE INVENTION

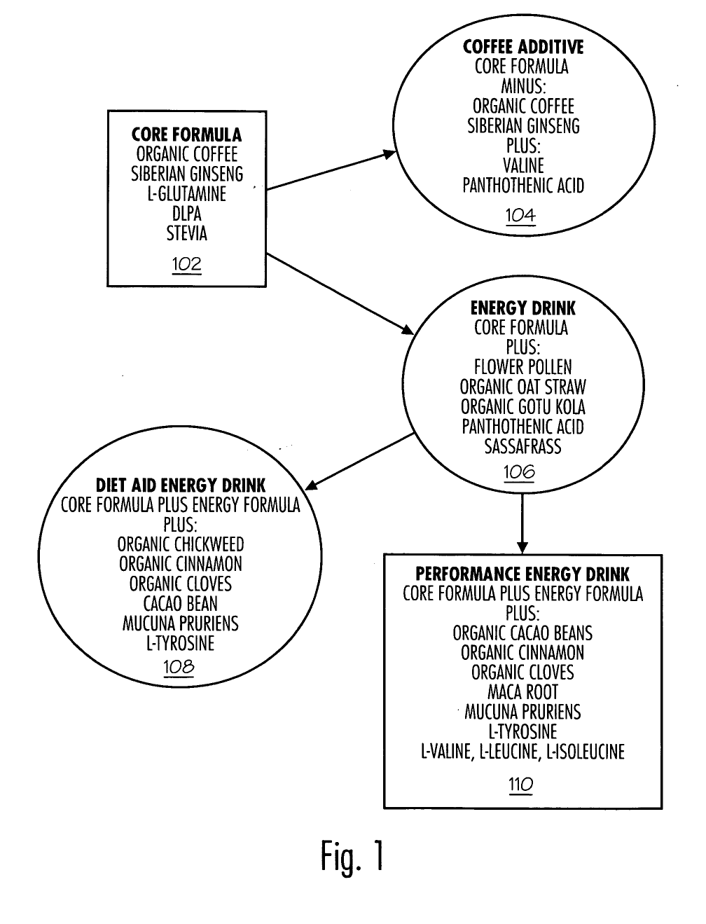

FIG. 1 is a chart illustrating the composition of some formulations of the present invention. In one embodiment, the present invention utilizes caffeine from a natural source to shuttle nutraceuticals of the drinks to the body. Nutraceuticals are any natural, bio-active chemical compound that has health promoting, disease preventing or medicinal properties. Examples of nutraceuticals include herbs, amino acids and vitamins. A type of nutraceuticals are nootropics. Nootropics are any nutraceuticals that performs neural enhancing function.

Core ingredient box 102 lists one embodiment of a core formulation of the present invention. The core (or base) formulation forms the basis for the drink compositions of the present invention. In the embodiment of FIG. 1, the core formula comprises organic coffee, Siberian ginseng, L-glutamine, DL-phenyalanine (DLPA) and stevia.

Organic coffee provides a natural source of caffeine. The caffeine serves to activate the brains noradrenaline stores. Noradrenaline is the brains version of adrenaline. Caffeine also activates dopamine-like substances in the brain. This promotes a general sense of well being in the drink. Another benefit of caffeine is that it is believed to shuttle nutraceuticals directly to the blood stream. This enhances the ability of the other ingredients to quickly get into the drinker's bloodstream. A natural, full spectrum source of caffeine is better than an isolated source, because the former contains all of the proportionate, synergistic co-factors necessary to deliver the caffeine in a balanced, harmonious manner. When you isolate caffeine, you have, in effect, created a drug that Nature never intended to be administered, without its accompanying botanical constituents. Much in the same way that Vitamin C is more effective when derived from whole plant extracts (and not synthetic ascorbic acid), caffeine is more gentle and healthy when absorbed from a natural plant source, such as guarana or organic coffee beans.

Siberian ginseng is an herb that has many beneficial effects. Ginseng helps to moderate the effects of stress. Ginseng also enhances physical and mental energy, endurance, stamina and performance. Ginseng is believed to increase resistance to disease and speed recuperation from illness. This ginseng can be considered a nootropic. The source of Siberian ginseng can be organic or wild crafted.

L-Glutamine is an amino acid. It is used by the body's muscles for metabolic energy (it is used in protein metabolism). L-Glutamine decomposes to form glutamic acid, which helps neutralize metabolic waste such as ammonia. L-Glutamine also produces GABA, an amino acid important for brain function. The breakdown of L-glutamine also produces glucose, a fuel source for the brain and skeletal muscles.

DL-Phenylalanine is an amino acid and is a building block in the body's process to create neurotransmitters known as catecholamines. Catecholamines include norepinephrine and noradrenaline. These catecholamines are normally secreted during the body's flight or fight response to stress and serve to increase alertness, stamina and strength in times of stress. Caffeine, such as the caffeine provided by organic coffee, helps to release catecholamines such as noradrenaline. DL-phenylalanine provides the raw material to produce more catecholamines. Thus, the DL-phenylalanine adds fuel to the body's fuel tank. DL-phenylalanine helps individuals to function at peak levels and to handle stress, danger and excitement. It also helps to provide relief from depression, and it elevates mood. DL-phenylalanine can be considered nootropic.

Stevia is a natural sweetener that comes from a shrub native to Paraguay and has been used by the indigenous Guarani Indians for thousands of years. Stevia is safe for use by diabetics. Because of Stevia's ability to regulate Pancreatic function, it assists with managing optimum blood glucose levels. Stevia may also help to reset the hunger mechanism of the Hypothalamus gland, thus supporting those persons with weight-loss and/or appetite concerns. In one embodiment, the stevia is refined white stevia powder. The stevia can also be in the form of unrefined powdered whole stevia leaf. In one embodiment, the drink comprises refined white stevia powder and unrefined powdered whole stevia leaf.

These ingredients of one embodiment of the core formulation form the basis of the other embodiments of drink formulations of the present invention. By themselves, these ingredients can be made into a drink that is beneficial to the drinkers. These core ingredients can be brewed like a tea or made into a ready to drink product that can be bottled and consumed at any time without further preparation. Additional ingredients, as detailed below, can be added to the core ingredients to produce other formulations.

In another embodiment of the present invention, a coffee additive is provided. The coffee additive comprises, in one embodiment, the core formula minus the organic coffee. In another embodiment, the coffee additive comprises L-glutamine, stevia, DL phenylalanine, pantothenic acid and L-valine as seen in box 104. The coffee additive can be mix with prepared coffee provided by the consumer. For example, the consumer may purchase coffee or brew coffee. This coffee additive supplementation turns regular coffee into a cognitive enhancing supplement and replenishes vital neurotransmitters depleted through caffeine consumption.

The drinks of the present invention can be made into a tea that can be brewed or as a ready made drink.

Energy Drink

The addition of other ingredients to the core ingredients or formula can add to the energy enhancing properties of the drink mix. As seen in box 106, in one embodiment of the present invention an energy drink is formed by the addition of flower pollen, organic oat straw, organic gotu kola, sasafrass and panthenic acid. The energy drink is designed to provide the drinker with higher levels of energy as well as mood and cognitive enhancements.

Flower pollen is the pollen grain from a flower. Flower pollen is a complex botanical comprising eighteen proteins, vitamins, sixteen minerals and trace minerals as well as essential enzymes and co-enzymes. Flower pollen is said to have a beneficial effect on the endocrine system and the nervous system. Flower pollen is useful in improving immune deficiencies and immune stress disorder.

Organic oat straw is the stalk portion of the plants whose seeds are milled and processed to produce oat flour and oatmeal. Oat straw is believed to be a nerve restorative that promotes clear thinking and helps to relieve depression. Thus, oat straw can be considered nootropic.

Gotu kola is an herb derived from a plant commonly known as the Indian Pennywort, which is indigenous to India, Sri Lanka, Madagascar, South Africa and the tropics. Gotu kola has been used to enhance concentration and memory and prevent mental fatigue. Thus, gotu kola can be considered a nootropic. It also increases energy and relaxes the body. Gotu kola is also a central nervous system stimulant and aids in circulation. The gotu kola is preferably organic.

Sasafrass comes from the root bark of the sasafrass tree. Sasafrass is believed to purify and stimulate the body's systems. It also is believed to help against fatigue. Sasafrass is also known to aid in the treatment of indigestion. In preferred embodiments, the source of sasafrass is organic or wild crafted.

Pantothenic acid is an anti-oxidant that increases stamina and helps the body respond to stress. Panothenic acid converts choline into acetylcholine, a neurotransmitter that facilitates intemeural cellular communication. Pantothenic acid can be considered a nootropic. Pantothenic acid also produces endogenous steroid hormones, which are useful when the body is stressed to help the body respond to the stress.

Mixed together with the core ingredients these additional ingredients are used to make an energy drink that helps to provide energy as well as replenishes the body's ability to produce energy. In one embodiment, the energy enhancing drink can be made into a tea. In another embodiment, the ingredients can be made into a ready made drink. In another embodiment, the energy enhancing formula minus the organic coffee can be provide as a coffee additive mix to add to coffee provided by the consumer.

Performance Drink

A combination of the core formula plus the energy drink ingredients plus other ingredients provides a performance enhancing drink as seen in box 110. In one embodiment, the performance drink comprises one or more additional ingredients added to the core formula plus the energy drink ingredients. The additional ingredients for the performance enhancing drink are selected from the group consisting of cinnamon, cloves, maca root, cacao beans, mucuna pruriens, L-tyrosine, L-valine, L-leucine, and L-isoleucine. In one embodiment, the performance enhancing drink consists of the core formula plus the energy enhancing additives plus cinnamon, cloves, -maca root, cacao beans, mucuna pruriens, L-tyrosine, L-valine, L-leucine, and L-isoleucine. The combination yields a performance drink to help both the professional athlete and the recreational athlete.

Cinnamon bark helps to fight fatigue. Cinnamon bark also believed to help to boost the effectiveness of other herbs. Cinnamon imparts a thermogenic effect in the body which helps to raise metabolic rate and increase warmth in the body's extremities. This botanical also assists with the absorption and conversion of carbohydrates. In one embodiment, the cinnamon is in the form of cinnamon chips. The cinnamon is preferably organic.

Maca Root is a perennial tuber grown high in the Peruvian Andes. Rich in iron, calcium and essential minerals, this root helps to balance menstrual irregularities, while also increasing fertility. It also works wonders for sterility and reproductive disorders in both sexes. Despite poor soil and a very harsh, arid environment, Maca thrives in the rarefied air above 14,000 feet. Perhaps, for this reason it is able to transfer the qualities of its hardy character and enhances endurance and stamina in humans and animals. With its high sterol profile, Maca is also taken by body-builders, as a potent alternative to anaboloic steroids. Maca energizes the body, helps to ameliorate chronic fatigue, perimenopausal and menopausal discomfort, enhances memory and balances hormonal secretions. The maca root is preferably organic. In one embodiment, the maca root is a powder.

Taking Mucuna pruriens stimulates the secretion of growth hormone (Hgh) by the pituitary gland. Growth hormone is undoubtedly the most powerful anti-ageing hormone: it encourages muscle mass and discourages body fat, improves stamina and energy levels, increases a sense of well-being and has a positive influence on many other aspects of health. Mucuna pruriens is also used in ayurvedic medicine, amongst others: to restore libido (in conjunction with Tribulus terrestris) by increasing levels of testosterone (as shown in a controlled study) and dopamine; in cases of male and female sterility (by increasing sperm count and encouraging ovulation), to improve mental alertness, motor coordination and to treat conditions of apathy. Mucuna pruriens is also known as velvet bean. It is preferably from an organic source. In one embodiment, the mucuna pruriens is a powder.

L-Tyrosine is important to the structure of almost all proteins in the body. This non-essential amino-acid is the precursor to several neurotransitters, most notably, noradrenaline, norepinephrine, L-Dopa and Dopamine. It is synthesized in the body from existing levels of phenylalanine. Environmental stress is associated with reduced levels of norepinephrine, as is physical stress, such as exercise. Because of its role in the production of key catecholoamines, L-Tyrosine may assist the body in coping with environmental, physical, emotional and mental stressors. The use of exogenous tyrosine has been conclusively shown in studies to transiently increase plasma and CNS catecholoamines, potentially making this amino-acid useful as an anti-depressant and anorexiant (appetite suppressant), while increasing memory and cognitive function under stress.

L-Leucine, L-isoleucine and L-valine are all essential amino acids and active forms of branched chain amino acids (BCAAs). They make up one-third of the muscle's protein levels, and stimulate protein synthesis directly in muscle tissue. Together they act to prevent muscle loss while increasing muscle mass, as well as promoting muscle recovery after physical exercise. L-Leucine has many beneficial effects on sports performance. It helps preserve lean muscle tissue, supplies the body with energy when under stress, preserves muscle glycogen (glucose stored in muscle tissue used to power muscular contraction), maintains nitrogen balance, and enhances thinking abilities that can decline as physical activity becomes more intense. L-Leucine, in particular, stimulates protein synthesis in the muscle, and the consumption of BCAAs (with 30 percent to 35 percent L-leucine) prior to and during endurance exercise has been found to prevent or decrease the net rate of protein degradation in muscle. L-Leucine also helps with the regulation of blood-sugar levels, the growth and repair of muscle tissue, growth hormone production, wound healing as well as energy regulation. It can assist in preventing the breakdown of muscle proteins that sometimes occur after trauma or severe stress. It may also be beneficial for individuals with phenylketonuria—a condition in which the body cannot metabolize the amino acid phenylalanine. Isoleucine, together with the other two branched-chain-amino-acids, promote muscle recovery after physical exercise, and on its own it is needed for the formation of hemoglobin as well as assisting with regulation of blood sugar levels as well as energy levels. It is also involved in blood-clot formation. L-Valine is a major fuel involved in anabolic (tissue-building) reactions, and stimulates muscle protein synthesis. A source of L-Leucine, L-isoleucine and L-valine is branch chain amino acid powder (BCAA powder). In one embodiment of the performance drink, the ratio of L-leucine to L-valine and L-isoleucine is 2:1.

Cloves are the buds off of the clove tree. Cloves are known to benefit the immune system. Cloves are also beneficial in the treatment of indigestion. The cloves are preferably whole and organic.

Cacao beans contain phytochemicals known as flavonoids. The flavonoids in cacao beans are believed to relax blood vessels and promote circulation. Organic Cacao Beans also contain significant amounts of trace minerals, which are all but depleted in most of the world's top-soil. The cacao beans can be used in the form of nibs.

Trace minerals, supplied in the body's natural ratios assist an athlete with recovery and help to improve performance. Another function of the cacao bean is its ability to help the body maintain proper pH levels. Cacao beans have long been revered amongst indigenous native cultures as an adrenal tonic, thus it is believed that the cacao bean can support the adrenal systems of those persons engaged in rigorous athletic activities.

Together with the core ingredients these additional ingredients are used to make as performance drink that replenishes the body's fuel. This performance drink provides, in addition to performance enhancing ingredients, needed trace minerals, vitamins, enzymes and amino acids. In one embodiment, the performance drink can be made into a tea. In addition, the ingredients can be made into a ready-made drink. In another embodiment, the performance enhancing formula minus the organic coffee can be provide as a coffee additive mix to add to coffee provided by the consumer.

Dietary Aid Energy Drink

A dietary aid drink can be made from the ingredients of the core formula plus the energy drink plus other ingredients as listed in box 108. The one or more additional ingredients are selected from maca root, cacao bean, mucuna pruriens (velvet bean), cinnamon, cloves, chickweed, and L-tyrosine. This formulation helps in adipoic (fat) tissue reduction, detoxification and the maintenance of an ideal weight. In one embodiment, the dietary aid comprises the core formula plus the energy drink ingredients plus maca root, cacao bean, mucuna pruriens, cinnamon, cloves, chickweed, and L-tyrosine. In a preferred embodiment, the dietary aid comprises the core formula plus the energy drink ingredients plus cacao bean, mucuna pruriens, cinnamon, cloves, chickweed, and L-tyrosine.

The benefits of cinnamon, cloves, maca root, cacao bean, mucuna pruriens and L-Tyrosine have already been discussed. Chickweed is believed to help remove toxins from the body, as well as act to reduce cellulite, systemically in the body. By eliminating toxic lymph congestion, Chickweed can effect a pronounced reduction in systemic cellulite. Furthermore, by stimulating the thyroid, Chickweed can promote additional weight loss in persons suffering from hypothyroidism. Chickweed also nourishes the blood tonifies stomach and spleen Qi, enhances nutrient absorption, and helps to eliminate stress-induced fatigue.

The core ingredients, the ingredients from the energy drink, and these additional ingredients can be used to make a skinny drink or dietary aid that replenishes the body's fuel. Also, unlike other dietary drinks, this formulation includes mood enhancing ingredients that help to uplift the mood of the drinker. In one embodiment this drink can be made into a tea. In addition, the ingredients can be made into a ready made drink. . In another embodiment, the diet aid formula minus the organic coffee can be provide as a coffee additive mix to add to coffee provided by the consumer.

Making the Tea Bags

In one embodiment, the different drinks are consumed as a tea. To facilitate the making of the tea, the ingredients are provided in a tea bag. The tea bag is preferably made form a flow-through mesh cotton. In order to achieve the proper dissolution of the nutraceuticals in the tea bag a process known as mass sequential layering is used to make the tea bag.

In mass sequential layering, the ingredients are placed in the tea bag in order of density and/or mass, starting with the largest mass materials and continuing with ingredients of smaller weights and consistency. For example, ingredients such as barks and roots are placed in the tea bag before ingredients such as leaves or flowers.

This prevents the denser ingredients from encapsulating or covering the less dense material. Instead the lighter materials are able to float freely to the top of the tea bag and infuse the heated water. The effect of loading all ingredients into layers is known as herbal stratification since placement of the herbs resembles sedimentary layers of rock.

Additionally, the present invention preferably utilizes coarsely ground herbs as opposed to powered herbs. Coarsely ground herbs are less likely to oxidize when in storage. This helps to maintain the potency of the herbs better. Coarsely ground herbs are also better for the steeping process and are easier to dissolute than powdered herbs.

In one embodiment, the tea bag for the energy drink contains the following ingredients, which are inserted into the teabag in the following order:
1 tbs organic coffee;
1 tsp organic or wild crafted Siberian ginseng;
¾ tsp organic or wild crafted Sassafras;
½ tsp organic oat straw;
½ tsp organic gotu kola;
½ tsp Flower pollen;
500 mg pantothenic acid;
500 mg DL-Phenyalanine;
2500 mg L-Glutamine; and
1/16^{th} tsp refined white stevia powder;
   where tsp is a teaspoon measure and tbs is a tablespoon measure. In another embodiment, a ¼ tsp unrefined powered whole stevia leaf is added before the refined white stevia powder.

In one embodiment, the teabag for the performance drink contains the following ingredients, which are inserted in the following order:
2 tbs organic coffee;
1 tsp organic or wild crafted Siberian ginseng;
1 ½ tsp organic cacao bean;
1 tsp organic or wild-crafted Sassafras;
¼ tsp organic cinnamon;
¼ tsp organic cloves or 2 whole cloves;
1¼ tsp Maca root;
1 tsp Mucuna Pruriens;
½ tsp organic oat straw;
½ tsp organic gotu kola;
1 tsp flower pollen;
750 mg pantothenic acid;
500 mg L-Tyrosine;
1000 mg DL-Phenyalanine;
2500 mg Branch Chain Amino Acid Powder (BCAA powder);
3000 mg L-Glutamine; and
½ to 1 tsp refined white stevia powder.

In one embodiment, the tea bag for the diet aid energy drink contains the following ingredients, which are placed in the following order:
1 tbs organic coffee;
1 tsp organic or wild crafted Siberian ginseng;
¾ tsp organic or wild crafted Sassafras;
¼ tsp organic cinnamon;
2 whole organic cloves;
½ tsp organic oat straw;
1 tsp organic gotu kola;
1 tbs Organic Chickweed;
½ tsp flower pollen;
500 mg Pantothenic acid;
1000 mg L-Tyrosine;
1000 mg DL-Phenyalanine;
3000 mg L-Glutamine; and
1 tsp refined white stevia powder.
   In another embodiment, 1 tsp of Organic cacao bean and 1.5 tsp of mucuna pruiens is added, in this order, before the cinnamon.
   Brewing a Tea

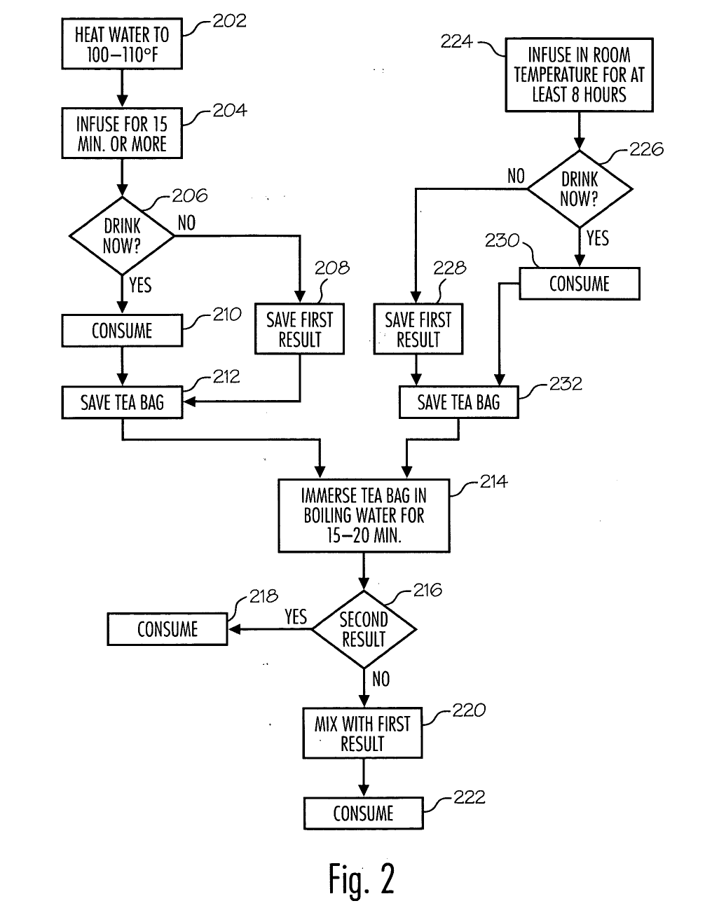

Once the tea bags are prepared, the drink is steeped and then consumed. In one embodiment the tea bag is infused in a two-part process. FIG. 2 is a flowchart illustrating the two-part process.

In the first part, step 202, 8-10 ounces of distilled or structured water is heated until it is hot to the touch, but well below the boiling point (approximately 100-110° F.). If the water temperature is too hot, the potency of some of the herbs may be affected. In a next step, step 204, the tea bag is placed in a cup or mug. The hot water is then poured over the tea bag. A lid is placed over the cup or mug to help maintain the proper steeping (infusing) temperature. This is then left to steep for at least fifteen minutes. At the end of the time, the infused water can be consumed as is (step 210) or saved for mixing with the product of the second part (step 208). In either case, the tea bag is saved (step 212).

In the second part, the same tea bag from part one is immersed in boiling water for fifteen to twenty minutes (step 214). Enough water is initially used to end with eight ounces of water after the boiling time. Alternatively, the tea bag can be placed in a thermos-type container, such as a steel travel mug, with boiling water for at least fifteen minutes. The resultant beverage can be consumed as is (step 218) or mixed with the beverage from part one (step 220) and consumed (step 222). The advantage of the two-part process is that higher heat and longer decoction times are needed for denser herbs, such as roots and barks. Conversely, aromatic herbs like flowers or herbs with volatile oils can be damaged by excessive heat and duration, while steeping. Another benefit to this two-step process is that the flower pollen's enzymatic potency is preserved by keeping temperature lower in the first steeping. This liquid is then put aside and combined with the more thoroughly decocted fluid later. As an alternative to part one, a cold infusion process can be used. In this case the tea bag is placed in room temperature, structured water for at least eight hours (step 224). The resultant beverage can be consumed as is (step 230) or saved to be mixed with the result of part two (step 228).

In another method of the present invention, the drinks of the present invention may be brewed using a covered pot. In the first step, eight ounces of coffee is brewed, using distilled or structured water. Preferably, the water is EON® structured water. About 2 tbs of organic coffee per serving is used (the minimum amount of coffee is about 1.5 tbs). In step 2, for the performance drink, Siberian ginseng, sassafras, cacao bean, cinnamon, cloves, oat straw, gotu kola are placed in a covered pot of 12 to 16 fluid ounces of gently boiling water (preferably EON®) and decoct for fifteen to twenty minutes. About eight ounces of fluid should remain. The mixture is allowed to cool to around 95° F. In step 3, once the mixture is warm (below 100° F., perferrable about 95° F.) the remaining ingredients, maca root powder, organic velvet bean powder, flower pollen, pantothenic acid, DL phenylalanine, L-tryosine, BCAA powder, L-glutamine and refined white stevia powder, are added and allowed to decoting for at least fifteen minutes. The liquid from step 3 can then be added to the coffee of step 1 and consumed. All neutraceutials should be soluble in the liquid. This process may also be used for the other drink formulation of the present invention. This method may be used to also prepare a ready to rink beverage.

In an alternative embodiment, a tea bag comprising the coffee additive mix is steeped in coffee supplied by the consumer. The coffee is preferably organic coffee.

Ready to Drink

The mixture can also be prepared as a ready to drink product. In this case, the ingredients are prepared commercially using structured water as a liquid base (hexagonally or tetrahedronally structured water is best, due to its stability and positive energetic spin. Pentagonal or five-molecule-per-cluster, structured water should not be used). This can then be sold and purchased in stores. This allows for the benefits of the formulations of the present invention to be provided in easy to use package. In one embodiment, the ready to drink formula includes:
16 ounces of hexagonally structured water;
2 tbs. Organic coffee;
1 tsp. Siberian ginseng;
1.5 tsp. Organic cacao beans;
½ tsp. Organic maca root;
1 tsp. Organic Mucuna Pruriens;
1 tsp. Wild-crafted Sassafras;
¼ tsp. Organic cinnamon;
2 Whole Organic cloves;
½ tsp. Organic oat straw;
1/2 tsp. Organic gotu kola;
1 tsp. Flower pollen;
750 mg. Pantothenic acid;
1000 mg. DL-Phenylalanine;
500 mg. L-Tyrosine;
2500 mg. Branch Chain Amino Acid Powder;
3000 mg. L-Glutamine; and
1/2 to 1 tsp. Refined, white, stevia powder

These ingredients are for a ready to drink performance drink.

Having now described preferred embodiments of the invention modifications and variations may occur to those skilled in the art. The invention is thus not limited to the preferred embodiments, but is instead set forth in the following clauses and legal equivalents thereof.

What is claimed:

1. A nutritional drink formulation comprising:
one or more nutraceuticals; each of the one or more nutraceuticals enhancing body functions, and a natural source of caffeine, the caffeine operable to assist the delivery of the nutraceuticals to the bloodstream.

2. The formulation of claim 1, wherein at least one of the nutraceuticals is a nootropic nutraceutical.

3. The formulation of claim 2, wherein the nootropic nutraceutical includes one or more of ginseng, L-Glutamine, and DL-Phenyalanine.

4. The formulation of claim 1, wherein the nutraceutical includes one or more of ginseng, L-Glutamine, and DL-Phenyalanine.

5. The formulation of claim 1, wherein the nutraceutical includes one or more of ginseng, L-Glutamine, and DL-Phenyalanine, and further comprising at least one or more additional ingredients selected from the group consisting of stevia, flower pollen, oat straw, gotu kola, sassafras, cacao beans, maca root, mucuna pruriens, cloves, cinnamon bark, chickweed, pantothenic acid, L-tyrisine, L-leucine, L-isoleucine and L-valine.

6. The formulation of claim 4, further comprising one or more of the flower pollen, oat straw, gotu kola, sassafras, pantothenic acid and stevia, to form an energy enhancing drink mix.

7. The formulation of claim 6, further include one or more of cinnamon bark, cloves, cacao beans, maca root, mucuna pruriens, L-Tyrosine, L-leucine, L-isoleucine and L-valine, to form a performance enhancing drink mix.

8. The formulation of claim 6, further comprising one or more of cinnamon bark, cloves, cacao beans, maca root, mucuna pruriens, L-Tyrosine and chickweed to form a dietary drink mix.

9. The formulation of claim 5, wherein the ingredients are provided in a coarsely ground form.

10. The formation of claim 9, wherein the ingredients are placed in a teabag according to the density of the ingredients.

11. The formulation of claim 10, wherein the teabag is steeped in a sufficient amount of water at 100-110° F. for at least fifteen minutes to form a first beverage.

12. The formulation of claim 11 wherein the teabag used to make the first beverage is boiled in water for fifteen to twenty minutes to form a second beverage.

13. The formulation of claim 12 wherein the first beverage and the second beverage are combined to make a third beverage.

14. The formulation of claim 9 wherein the teabag is infused in room temperate water for at least eight hours to form a fourth beverage.

15. The formulation of claim 5, wherein the ready to drink is prepared as ready to drink beverage.

16. A nutritional drink formulation comprising:
one or more nutraceuticals; each of the one or more nutraceuticals enhancing body functions, and wherein the nutraceutials is selected from the group consisting of ginseng, L-Glutamine, and DL-Phenyalanine;
a natural source of caffeine, the caffeine operable to assist the delivery of the nutraceuticals to the bloodstream; and
and one or more additional ingredients selected from the group consisting of stevia, flower pollen, oat straw, gotu kola, sassafras, cacao beans, maca root, mucuna pruriens, cloves, cinnamon bark, chickweed, pantothenic acid, L-tyrisine, L-leucine, L-isoleucine and L-valine.

17. The drink formulation of claim 16, wherein the formulation is provided as a tea to be brewed.

18. The drink formulation of claim 17, wherein the tea is brewed with distilled or structured water.

19. The drink formulation of claim 16, wherein the formulation is a ready to drink beverage.

20. A coffee additive for adding to brewed coffee comprising L-gutamine, DL-Phenyalanine, stevia L-valine and pantothenic acid.
